(19) United States
(12) Patent Application Publication
(10) Pub. No.: US 2002/0119114 A1
(43) Pub. Date: Aug. 29, 2002

(54) Active ingredient combinations of surface-active citric esters and alpha-lipoic acid, and cosmetic and dermatological preparations containing such mixtures

(75) Inventors: Thomas Raschke, Hamburg (DE); Inge Kruse, Hamburg (DE); Uwe Schonrock, Nahe (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Beiersdorf Aktiengesellschaft

(21) Appl. No.: 10/021,217

(22) Filed: Dec. 12, 2001

(30) Foreign Application Priority Data
Dec. 18, 2000 (DE) 100 63 129.0

(51) Int. Cl. A61K 7/075; A61K 7/08

(52) U.S. Cl. 424/70.22

(57) ABSTRACT

Combinations of:

(a) one or more partially neutralized esters of monoglycerides and/or diglycerides of saturated fatty acids with citric acid; and

(b) α-lipoic acid.

The present invention relates to active ingredient combinations of surface-active citric acids and α-lipoic acid, and to cosmetic and dermatological preparations containing such mixtures, and to the use of surface-active citric acid esters of stabilizing α-lipoic acid against chemical degradation reactions, in particular photochemical degradation reactions and/or oxidation-induced degradation reactions.

Moreover, the invention relates to synergistic mixtures of α-lipoic acid and surface-active substances, and to cosmetic and dermatological preparations containing such mixtures. The present invention preferably relates to cosmetic preparations with effective protection against harmful oxidation processes in the skin, but also for the protection of cosmetic preparations themselves or for the protection of the constituents of cosmetic preparations against harmful oxidation processes.

The present invention accordingly relates, in preferred embodiments, to cosmetic or dermatological preparations comprising active ingredients for the care and protection of the skin, in particular sensitive skin and, very particularly, skin aging or aged by intrinsic and/or extrinsic factors, and to the use of such active ingredients and combinations of such active ingredients in the field of cosmetic and dermatological skincare.

The human skin is man's largest organ and performs a number of vital functions. Having an average area of approximately 2 m² in adults, it has a prominent role as a protective and sensory organ. The purpose of this organ is to transmit and avert mechanical, thermal, actinic, chemical and biological stimuli. In addition, it has an important role as a regulatory and target organ in human metabolism.

The main aim of skincare in the cosmetics sense is to strengthen or restore the skin's natural function as a barrier against environmental influences (e.g. dirt, chemicals, microorganisms) and against the loss of substances intrinsic to the body (e.g. water, natural fats, electrolytes), and also to assist its horny layer in its natural regeneration ability in cases of existing damage.

Impairment of the barrier properties of the skin may lead to increased resorption of toxic or allergenic substances or to attack by microorganisms, leading to toxic or allergic skin reactions.

Another aim of skincare is to compensate for the loss by the skin of sebum and water caused by daily washing. This is particularly important if the natural regeneration ability is inadequate. Furthermore, skincare products should protect against environmental influences, in particular against sun and wind, and delay skin aging.

Chronological skin aging is caused, for example, by endogenous genetically determined factors. The following structural damage and functional disorders, which can also fall under the term “senile xerosis”, arise, for example, in the epidermis and dermis as a result of aging:

a) dryness, roughness and formation of dryness wrinkles,

b) itching and

c) reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders arise in the skin in particular as a result of exogenous factors; these are more far-reaching than the degree and quality of the damage in the case of chronological aging:

d) visible vascular dilation (telangiectases, couperosis);

e) flaccidity and formation of wrinkles;

f) local hyperpigmentation, hypopigmentation and abnormal pigmentation (e.g. age spots) and

g) increased susceptibility to mechanical stress (e.g. cracking).

The present invention relates in particular to products for the care of skin aged naturally, and to the treatment of the damage caused by photoaging, in particular of the phenomena listed under a) to g).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development there are considerable difficulties in stabilizing the active ingredients to an adequate extent against oxidative decay. The use of products comprising vitamin A acid, moreover, often causes severe erythematous skin irritations. Retinoids can therefore only be used in low concentrations.

In particular, the present invention relates to cosmetic preparations having effective protection against harmful oxidation processes in the skin, but also for the protection of cosmetic preparations themselves or for the protection of the constituents of cosmetic preparations against harmful oxidation processes.

The present invention further relates to antioxidants, preferably those used in skincare cosmetic or dermatological preparations. In particular, the invention also relates to cosmetic and dermatological preparations comprising such antioxidants. In a preferred embodiment, the present invention relates to cosmetic and dermatological preparations for the prophylaxis and treatment of cosmetic or dermatological skin changes, such as, for example, skin aging, in particular skin aging caused by oxidative processes.

Furthermore, the present invention relates to active ingredients and preparations comprising such active ingredients for the cosmetic and dermatological treatment or prophylaxis of erythematous, inflammatory, allergic or autoimmune-reactive symptoms, in particular dermatoses.

In a further advantageous embodiment, the present invention relates to active ingredient combinations and preparations which serve for the prophylaxis and treatment of light-sensitive skin, in particular of photodermatoses.

The harmful effect of the ultraviolet part of solar radiation on the skin is generally known. Whereas rays with a wavelength of less than 290 nm (the UVC region) are absorbed by the ozone layer in the earth's atmosphere, rays in the range between 290 nm and 320 nm, the UVB region, cause erythema, simple sunburn or even burns of greater or lesser severity.

A maximum erythema activity of sunlight is given as the relatively narrow range around 308 nm.

Numerous compounds are known for protecting against UVB radiation; these are derivatives of 3-benzylidenecamphor, of 4-aminobenzoic acid, of cinnamic acid, of salicylic acid, of benzophenone and also of 2-phenylbenzimidazole.

It is also important to have available filter substances for the range between about 320 nm and about 400 nm, the UVA region, since its rays can cause reactions in cases of photosensitive skin. It has been found that UVA radiation leads to damage of the elastic and collagenous fibers of connective tissue, which leads to premature aging of the skin, and is to be regarded as a cause of numerous phototoxic and photoallergic reactions. The harmful effect of UVB radiation can be intensified by UVA radiation.

To protect against rays of the UVA region, therefore, certain derivatives of dibenzoylmethane are used, the photostability of which is inadequate (Int. J. Cosm. Science 10, 53 (1988)).

The UV radiation can, however, also lead to photochemical reactions, in which case the photochemical reaction products then intervene in the skin's metabolism.

Such photochemical reaction products are predominantly free-radical compounds, for example hydroxyl radicals. Undefined free-radical photoproducts which form in the skin itself can also display uncontrolled secondary reactions because of their high reactivity. However, singlet oxygen, a non-free-radical excited state of the oxygen molecule, can also be formed during UV irradiation, as can short-lived epoxides and many others. Singlet oxygen, for example, differs from normal triplet oxygen (free-radical ground state) by virtue of its increased reactivity. However, excited, reactive (free-radical) triplet states of the oxygen molecule also exist.

UV radiation is also a type of ionizing radiation. There is therefore the risk that ionic species will also form during UV exposure, which then for their part are able to intervene oxidatively in the biochemical processes.

In order to prevent these reactions, additional antioxidants and/or free-radical scavengers can be incorporated into the cosmetic or dermatological formulations.

It has already been proposed to use vitamin E, a substance with known antioxidative action, in light protection formulations, although, here too, the effect achieved falls a long way short of expectations.

The object of the invention was therefore to provide cosmetic, dermatological and pharmaceutical active ingredients and preparations, and light protection formulations which serve for the prophylaxis and treatment of photosensitive skin, in particular photodermatoses, preferably PLD.

Other names for polymorphous photodermatosis are PLD, PLE, Mallorca acne and a large number of other names, as given in the literature (e.g. A. Voelckel et al, Zentralblatt Haut-und Geschlechtskrankheiten (1989), 156, p.2).

Erythematous skin symptoms also occur as accompanying symptoms in certain skin diseases or irregularities. For example, the typical skin rash symptom of acne is generally red to a greater or lesser extent.

Antioxidants are mainly used as substances which protect against the deterioration of the preparations in which they are present. Nevertheless, it is known that in human or animal skin as well, undesired oxidation processes may occur. Such processes play an important role in skin aging.

The essay “Skin Diseases Associated with Oxidative Injury” in “Oxidative Stress in Dermatology”, p. 323 ff. (Marcel Decker Inc., New York, Basel, Hong Kong, Editor: Jürgen Fuchs, Frankfurt, and Lester Packer, Berkeley/California) discusses oxidative skin damage and its more obvious causes.

Also for the reason of preventing such reactions, antioxidants and/or free-radical scavengers can be additionally incorporated into cosmetic or dermatological formulations.

A number of antioxidants and free-radical scavengers are known. For example U.S. Pat. No. Specifications 4,144,325 and 4,248,861, and numerous other documents have already proposed the use of vitamin E, a substance with known antioxidative action in light protection formulations, although here too the effect achieved falls a long way short of the desired effect.

Customary cosmetic administration forms are emulsions. This term generally means a heterogeneous system of two liquids which are immiscible or miscible only to a limited extent with each other, which are usually referred to as phases. One is in the form of droplets (disperse or internal phase), while the other liquid forms a continuous (coherent or internal) phase. Less common administration forms are multiple emulsions, i.e. those which, in the droplets of the dispersed (or discontinuous) phase, comprise for their part droplets of a further dispersed phase, e.g. W/O/W emulsions and O/W/O emulsions.

More recent findings have recently lead to a better understanding of cosmetic emulsions which are relevant in practice. Here, it is assumed that the emulsifier mixtures used in excess form lamellar liquid-crystalline phases or crystalline gel phases. In the gel network theory, stability and physicochemical properties of such emulsions are attributed to the formation of viscoelastic gel networks.

If the two liquids are water and oil and the oil droplets are finely dispersed in water, then this is an oil-in-water emulsion (O/W emulsion, e.g. milk). The basic character of a O/W emulsion is defined by the water. In the case of a water-in-oil emulsion (W/O emulsion, e.g. butter), the principle is reversed, the basic character being determined here by the oil.

In order to be able to ensure the metastability of emulsions, interface-active substances, i.e. emulsifiers, are usually necessary. The use per se of customary cosmetic emulsifiers is entirely acceptable. Nevertheless, emulsifiers, as ultimately any chemical substance, may in certain cases cause allergic reactions or reactions based on oversensitivity of the user. For example, it is known that in some particularly sensitive people, certain light dermatoses are triggered by certain emulsifiers and simultaneous action of sunlight.

It is possible to prepare emulsifier-free preparations which, for example, have, in an aqueous phase, dispersed oil droplets, similar to an O/W emulsion. A prerequisite may be that the continuous aqueous phase has a gel framework which stabilizes the dispersed phase, and other conditions besides. Such systems are sometimes called hydrodispersions or oleodispersions depending on which is the disperse phase and which is the continuous phase.

However, for cosmetic technology, it is neither necessary nor possible to dispense with emulsifiers entirely, especially since there is a certain choice of particularly mild emulsifiers. However, the prior art lacks a satisfactorily broad range of those emulsifiers which would then also significantly broaden the application spectrum of correspondingly mild cosmetic preparations which are tolerated by the skin.

An object of the present invention was therefore to provide cosmetic or dermatological preparations with excellent skincare properties.

α-Lipoic acid was isolated in 1952 from liver tissue and its structure was explained as a sulfur-containing fatty acid. Bacteria, plants and higher organisms can produce α-lipoic acid themselves in their metabolism; the question of whether humans biosynthesize their own α-lipoic acid is still open.

α-Lipoic acid is used in the therapy of polyneuropathy, a sensibility disorder in the hands and feet as a long-term effect of diabetes. 200 to 600 milligrams of α-lipoic acid per day lead to a significant reduction in pain intensity. The energy metabolism of the nerves in the hands and feet is activated by α-lipoic acid, resulting in better nerve conductivity and thus fewer feelings of numbness and reflex losses.

α-Lipoic acid reduces pathologically increased liver function values and promotes the healing of hepatitis. α-Lipoic acid is present in most foods in small amounts, relatively high contents only being found in meat. It is recognized that α-lipoic acid has strongly antioxidative properties.

α-Lipoic acid is characterized by the following chemical structure:

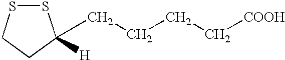

WO97/10808 and U.S. Pat. No. 5,472,698 describe the cosmetic use of α-lipoic acid against symptoms of skin aging. DE-42 42 876 describes active ingredient combinations of biotin and antioxidants with α-lipoic acid for the cosmetic and/or dermatological care of the skin and/or skin appendages, and also cosmetic and/or dermatological preparations comprising such active ingredient combinations.

It was therefore surprising and could not have been foreseen by the person skilled in the art that combinations of

(a) one or more partially neutralized esters of monoglycerides and/or diglycerides of saturated fatty acids with citric acid

(b) α-lipoic acid,

leads to preparations which are stable toward chemical degradation reactions, in particular photochemical degradation reactions and/or oxidation-induced degradation reactions, which increase the bioavailability of the α-lipoic acid whose effectiveness increases in a synergistic manner and thus overcome the disadvantages of the prior art.

A particularly advantageous partially neutralized ester of monoglycerides and/or diglycerides of saturated fatty acids with citric acid is glyceryl stearate citrate. Such citric esters are available, for example, under the product name “IMWITOR® 370” from Condea Chemie GmbH or “Axol C 62” from Goldschmidt AG.

The total amount of α-lipoic is advantageously chosen from the range 0.0001-10% by weight, preferably 0.005-5.0% by weight, in particular 0.01-3.0% by weight, based on the total weight of the formulation.

The total amount of the partially neutralized esters of monoglycerides and/or diglycerides of saturated fatty acids with citric acid used according to the invention in the finished cosmetic or dermatological preparations is advantageously chosen from the range 0.1-20% by weight, preferably 0.5-10.0% by weight, in particular 1.0-5.0% by weight, based on the total weight of the preparations.

It is advantageous to choose weight ratios between α-lipoic acid on the one hand and at least one partially neutralized ester of monoglycerides and/or diglycerides of saturated fatty acids with citric acid on the other hand from the range from 1:1 to 1:500, preferably 1:10 to 1:200, in particular 1:50.

The novel combination of α-lipoic acid and at least one partially neutralized ester of monoglycerides and/or diglycerides of saturated fatty acids with citric acid is also referred to for the purposes of this specification in collective terms as “active ingredients according to the invention” or “active ingredient used according to the invention” or “active ingredient combination used according to the invention” or is given synonymous designations.

The active ingredient combinations according to the invention can be incorporated without problems into customary cosmetic preparations, advantageously light protection preparations, but also, if desired, other preparations, for example pharmaceutical preparations.

The use of the active ingredient used according to the invention or of cosmetic or topical dermatological preparations with an effective content of active ingredient used according to the invention surprisingly enables effective treatment, but also prophylaxis

of deficient, sensitive or hypoactive skin states or deficient, sensitive or hypoactive states of skin appendages,

of symptoms of premature aging of the skin (e.g. wrinkles, age spots, telangiectases) and/or of the skin appendages,

of environmentally induced changes in the skin and the skin appendages (smoking, smog, reactive oxygen species, free radicals) and in particular light-induced negative changes,

of dry skin,

of light-induced skin damage,

of pigmentation disorders,

of irritation,

of dry skin conditions and impairment of the horny layer barrier,

of hair loss and for improved hair growth,

of inflammatory skin conditions, such as atopic eczema, seborrhoeic eczema, polymorphous photodermatosis, psoriasis, vitiligo.

The active ingredient according to the invention or cosmetic or topical dermatological preparations with an effective content of active ingredient according to the invention, however, also surprisingly serves

to calm sensitive or irritated skin,

to stimulate the synthesis of collagen, hyaluronic acid and elastin,

to stimulate intracellular DNA synthesis, in particular in cases of deficient or hypoactive skin states,

to increase cell renewal and regeneration of the skin,

to increase the skin's own protective and repair mechanisms (for example for dysfunctional enzymes, DNA, lipids, proteins),

for the pre- and post-treatment in cases of topical application of laser and abrasive treatments, which serve, for example, to reduce skin wrinkles and scars, to counteract the resulting skin irritations and to promote the regeneration processes in the damaged skin.

In particular, according to the invention, it is extremely advantageous to use the active ingredient used according to the invention or cosmetic or topical dermatological preparations with an effective content of active ingredient used according to the invention for the cosmetic or dermatological treatment or prophylaxis of undesired skin conditions.

The total amount of α-lipoic acid in the finished cosmetic or dermatological preparations is advantageously chosen from the range 0.025-10.0% by weight, preferably 0.1-5.0% by weight, based on the total weight of the preparations.

It could therefore not have been foreseen by the person skilled in the art that the active ingredient combinations used according to the invention or cosmetic or dermatological preparations comprising such combinations

would be more effective antioxidants

would be more effective free-radical scavengers

would better prevent the binding of harmful photoproducts to lipids, DNA and proteins

would better counter skin aging

would better protect the skin against photoreactions

would better prevent inflammatory reactions than the active ingredients, active ingredient combinations and preparations of the prior art. In addition, it could have not have been foreseen that the active ingredient combinations used according to the invention in cosmetic or dermatological preparations have higher stability than each of the active ingredients used individually, which relates in particular to α-lipoic acid.

The invention therefore provides for the use of active ingredient combinations of α-lipoic acid and at least one partially neutralized ester of monoglycerides and/or diglycerides of saturated fatty acids with citric acid as antioxidant and its use for the treatment and/or prophylaxis of skin aging caused by oxidative stress, and of inflammatory reactions.

In addition, a particularly advantageous embodiment of the present invention is regarded as being the use of active ingredient combinations of α-lipoic acid and at least one partially neutralized ester of monoglycerides and/or diglycerides of saturated fatty acids with citric acid for the treatment and/or prophylaxis of oxidative stress.

According to the invention, the cosmetic or dermatological preparations can have the customary composition and can be used for the treatment, care and cleansing of the skin and/or the hair and as a make-up product in decorative cosmetics. They preferably comprise 0.1% by weight to 20% by weight, preferably 0.5% by weight to 10% by weight, in particular 1.0-5.0% by weight, based on the total weight of the preparations, of active ingredient combinations used according to the invention.

According to the invention, it is preferred to add complexing agents to the active ingredient combinations used according to the invention or to cosmetic or dermatological preparations comprising such active ingredient combinations.

Complexing agents are auxiliaries used in cosmetology or medicinal pharmaceutical technology which are known per se. By complexing undesired metals such as Mn, Fe, Cu and others, it is possible, for example, to prevent undesired chemical reactions in cosmetic or dermatological preparations.

Complexing agents, in particular chelating agents, form complexes with metal atoms. In the presence of one or more polybasic complexing agents, i.e. chelating agents, the complexes are metallacycles. Chelates are compounds in which a single ligand occupies more than one coordination site on a central atom. In this case, normally extended compounds are thus closed as a result of complex formations via a metal atom or a metal ion to form rings. The number of bonded ligands depends on the coordination number of the central metal. A prerequisite for formation of the chelate is that the compound reacting with the metal contains two or more atomic groups which act as electron donors.

The complexing agent(s) can advantageously be chosen from the group of customary compounds, at least one substance preferably being chosen from the group consisting of tartaric acid and anions thereof, citric acid and anions thereof, aminopolycarboxylic acids and anions thereof (such as, for example, ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof).

According to the invention, the complexing agent(s) is/are advantageously present in cosmetic or dermatological preparations preferably in an amount of 0.001% by weight to 10% by weight, preferably in an amount of 0.01% by weight to 5% by weight, particularly preferably in an amount of 0.05-2.0% by weight, based on the total weight of the preparations.

For use, the cosmetic and dermatological preparations are, according to the invention, applied to the skin and/or the hair in an adequate amount in the manner customary for cosmetics.

According to the invention, cosmetic and dermatological preparations can be in various forms. It is particularly advantageous if they are an emulsion or a microemulsion of the oil-in-water (O/W) type.

It is also possible and advantageous for the purposes of the present invention to add active ingredient combinations used according to the invention to aqueous systems or surfactant preparations for cleansing the skin and the hair.

According to the invention, the cosmetic and dermatological preparations can comprise cosmetic auxiliaries as are customarily used in such preparations, e.g. preservatives, bactericides, perfumes, antifoams, dyes, pigments which have a coloring action, thickeners, surface-active substances, emulsifiers, emollients, moisturizers and/or hermectants, fats, oils, waxes, or other customary constituents of a cosmetic or dermatological formulation such as alcohols, polyols, polymers, foam stabilizers, electrolytes, organic solvents or silicone derivatives.

In particular, active ingredient combinations used according to the invention can also be combined with other antioxidants and/or free-radical scavengers.

Such antioxidants are advantageously chosen from the group consisting of amino acids (for example glycine, histidine, tyrosine, tryptophan) and derivatives thereof, imidazoles (for example urocanic acid) and derivatives thereof, peptides such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, pyridoxine and derivatives thereof, ubiquinone and ubiquinol and derivatives thereof, tocopherols and derivatives (for example vitamin E acetate), vitamin A and derivatives (vitamin A palmitate) and coniferyl benzoate of benzoin resin, rutinic acid and derivatives thereof, ascorbic acid and derivatives thereof, in particular ascorbyl palmitate, ascorbyl phosphate and related compounds, butylhydroxytoluene, butylhydroxyanisole, nordihydroguaiacic acid, nordihydroguaiaretic acid, trihydroxybutyrophenone, uric acid and derivatives thereof, mannose and derivatives thereof, sesamol, sesamolin, zinc and derivatives thereof (for example ZnO, ZnSO₄), selenium and derivatives thereof (for example selenomethionine), stilbenes and derivatives thereof (for example stilbene oxide, trans-stilbene oxide) and the derivatives of these active ingredients which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.025-20% by weight, in particular 0.05-10% by weight, based on the total weight of the preparation.

If ascorbic acid and/or derivatives thereof are the additional antioxidant(s), it is advantageous to choose the respective concentrations thereof from the range 0.001-10% by weight, based on the total weight of the formulation.

If vitamin E and/or derivatives thereof are the additional antioxidants, it is advantageous to choose the respective concentrations thereof from the range 0.001-10% by weight, based on the total weight of the preparation.

According to the invention, emulsions are advantageous and comprise, for example, said fats, oils, waxes and other fatty substances, and also water and an emulsifier, as is customarily used for this type of formulation.

The lipid phase can advantageously be chosen from the following group of substances:

mineral oils, mineral waxes;

oils, such as triglycerides of capric or of caprylic acid, also natural oils such as, for example, castor oil;

fats, waxes and other natural and synthetic fatty substances, preferably esters of fatty acids with alcohols of low C number, for example with isopropanol, propylene glycol or glycerol, or esters of fatty alcohols with alkanoic acids of low C number or with fatty acids;

alkyl benzoates;

silicone oils, such as dimethylpolysiloxanes, diethylpolysiloxanes, diphenylpolysiloxanes and mixed forms thereof.

For the purposes of the present invention, the oil phase of the emulsions, oleogels and hydrodispersions or lipodispersions is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then be advantageously chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, from the group of saturated or unsaturated, branched or unbranched alcohols, and also fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can advantageously be chosen, for example, from the group of synthetic, semisynthetic and natural oils, e.g. olive oil, sunflower oil, soybean oil, groundnut oil, rapeseed oil, almond oil, palm oil, coconut oil, palm kernel oil and the like.

For the purposes of the present invention, any mixtures of such oil and wax components can also advantageously be used. When required, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride and dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and isotridecyl isononanoate are particularly advantageous.

For the purposes of the present invention, of the hydrocarbons, paraffin oil, squalane and squalene can advantageously be used.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously used as the silicone oil to be used according to the invention. However, other silicone oils can also be advantageously used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are also particularly advantageous.

If appropriate, the aqueous phase of the preparations according to the invention advantageously comprises alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propanediol and glycerol, and, in particular, one or more thickeners, which can advantageously be chosen from the group consisting of silicon dioxide, aluminum silicates, polysaccharides and derivatives thereof, for example hyaluronic acid, xanthan gum and hydroxypropylmethylcellulose, particularly advantageously from the group of polyacrylates, preferably a polyacrylate from the group of Carbopols, for example Carbopol grades 980, 981, 1382, 2984 and 5984, or from the group of Pemulens, for example Pemulen grades TR-1 and TR-2, in each case individually or in combination.

In particular, mixtures of the abovementioned solvents are used. In the case of alcoholic solvents, water may be a further constituent.

Emulsions according to the invention advantageously comprise, for example, said fats, oils, waxes and other fatty substances, and also water and optionally one or more further emulsifiers, as is customarily used for this type of formulation.

Preparations according to the invention which are in the form of emulsions optionally particularly advantageously comprise one or more additional O/W emulsifiers. Such O/W emulsifiers can, for example, advantageously be chosen from the group of polyethoxylated or polypropoxylated or polyethoxylated and polypropoxylated products, e.g.:

fatty alcohol ethoxylates,

ethoxylated wool wax alcohols,

polyethylene glycol ethers of the general formula R—O—(—CH₂—CH₂—O—)ₙ—R',

fatty acid ethoxylates of the general formula R—COO—(—CH₂—CH₂—O—)ₙ—H,

etherified fatty acid ethoxylates of the general formula R—COO—(—CH₂—CH₂—O—)ₙ—R',

esterified fatty acid ethoxylates of the general formula R—COO—(—CH₂—CH₂—O—)ₙ—C(O)—R',

polyethylene glycol glycerol fatty acid esters,

ethoxylated sorbitan esters,

cholesterol ethoxylates,

ethoxylated triglycerides,

alkyl ether carboxylic acids of the general formula R—O—(—CH₂—CH₂—O—)ₙ—CH₂—COOH where n is a number from 5 to 30,

polyoxyethylene sorbitol fatty acid esters,

alkyl ether sulfates of the general formula R—O—(—CH₂—CH₂—O—)ₙ—SO₃—H,

fatty alcohol propoxylates of the general formula R—O—(—CH₂—CH(CH₃)—O—)ₙ—H,

polypropylene glycol ethers of the general formula R—O—(—CH₂—CH(CH₃)—O—)ₙ—R',

propoxylated wool wax alcohols,

etherified fatty acid propoxylates R—COO—(—CH₂—CH(CH₃)—O—)ₙ—R',

esterified fatty acid propoxylates of the general formula R—COO—(—CH₂—CH(CH₃)—O—)ₙ—C(O)—R',

fatty acid propoxylates of the general formula R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula R—O—(—CH₂—CH(CH₃)—O—)ₙ—CH₂—COOH,

alkyl ether sulfates or the parent acids of these sulfates of the general formula R—O—(—CH₂—CH(CH₃)—O—)ₙ—SO₃—H,

fatty alcohol ethoxylates/propoxylates of the general formula R—O—Xₙ—Yₘ—H,

polypropylene glycol ethers of the general formula R—O—Xₙ—Yₘ—R',

etherified fatty acid propoxylates of the general formula R—COO—Xₙ—Yₘ—R',

fatty acid ethoxylates/propoxylates of the general formula R—COO—Xₙ—Yₘ—H.

According to the invention, the polyethoxylated or polypropoxylated or polyethoxylated and polypropoxylated O/W emulsifiers used are particularly advantageously chosen from the group of substances having HLB values of 11-18, very particularly advantageously having HLB values of 14.5-15.5, if the O/W emulsifiers have saturated radicals R and R'. If the O/W emulsifiers have unsaturated radicals R and/or R', or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20),

polyethylene glycol (12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20),

polyethylene glycol(13) cetyl ether (ceteth-13), polyethylene glycol(14) cetyl ether (ceteth-14), polyethylene glycol(15) cetyl ether (ceteth-15 ), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20),

polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetylether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20),

polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15),

polyethylene glycol(12) lauryl ether (laureth-12), polyethylene glycol(12) isolauryl ether (isolaureth-12),

polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate,

polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate,

polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

Sodium laureth-11 carboxylate can advantageously be used as the ethoxylated alkyl ether carboxylic acid or salt thereof.

Sodium laureth-14 sulfate can advantageously be used as alkyl ether sulfate.

Polyethylene glycol(30) cholesteryl ether can advantageously be used as ethoxylated cholesterol derivative. Polyethylene glycol(25) soyasterol has also proven successful.

The polyethylene glycol(60) evening primrose glycerides can advantageously be used as ethoxylated triglycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethyleneglycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

According to the invention, preparations in the form of emulsions may, however, also optionally advantageously comprise one or more additional W/O emulsifiers. Such advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, glyceryl monocaprylate.

Gels according to the invention usually comprise alcohols of low carbon number, e.g. ethanol, isopropanol, 1,2-propanediol, glycerol and water or an abovementioned oil in the presence of a thickener which, in the case of oily alcoholic gels, is preferably silicon dioxide or an aluminum silicate, and, in the case of aqueous-alcoholic or alcoholic gels, is preferably a polyacrylate.

Preparations according to the invention can further advantageously comprise substances which absorb UV radiation in the UVB region, the total amount of filter substances being, for example, 0.1% by weight to 30% by weight, preferably 0.5 to 10% by weight, in particular 1.0 to 6.0% by weight, based on the total weight of the preparations, in order to make available cosmetic preparations which protect the hair or the skin from the entire range of ultraviolet radiation. They can also serve as sunscreens for hair or skin.

If the preparations according to the invention comprise UVB filter substances, these may be oil-soluble or water-soluble. Advantageous oil-soluble UVB filter substances are, for example:

3-benzylidenecamphor derivatives, preferably 3-(4-methylbenzylidene)camphor and 3-benzylidenecamphor;

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate,

derivatives of benzophenone, preferably 2-hydroxy-4-methoxybenzophenone, 2-hydroxy-4-methoxy-4′-methylbenzophenone and 2,2′-dihydroxy-4-methoxybenzophenone;

esters of benzalmalonic acid, preferably di(2-ethylhexyl) 4-methoxybenzalmalonate and

2,4,6-tris(p-2-ethylhexoxy carbonylanilino)-1,3,5-triazine.

Advantageous water-soluble UVB filters are, for example:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl) benzenesulfonic acid, 2-methyl-5-(2-oxo-3-bornylidenemethyl)sulfonic acid and salts thereof, and also 1,4-di(2-oxo-10-sulfo-3-bornylidenemethyl)benzene and salts thereof (the corresponding 10-sulfato compounds, for example the corresponding sodium, potassium or triethanolammonium salt) also referred to as benzene-1,4-di(2-oxo-3-bornylidenemethyl)-10-sulfonic acid.

The list of the UVB filters mentioned, which can be used in combination with the active ingredient combinations according to the invention, is not of course intended to be limiting.

The invention also provides for the use of a combination of the active ingredient combinations used according to the invention with at least one UVB filter as an antioxidant and for the use of a combination of the active ingredient combinations used according to the invention with at least one UVB filter as an antioxidant in a cosmetic or dermatological preparation.

It can also be advantageous to combine the active ingredient combinations used according to the invention with UVA filters which have to date customarily been present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations and preparations comprising these combinations are also provided by the invention. The amounts used are as for the UVB combination.

The invention also provides for the use of a combination of active ingredient combinations used according to the invention with at least one UVA filter as an antioxidant and for the use of a combination of the active ingredient combinations according to the invention with at least one UVA filter as an antioxidant in a cosmetic or dermatological preparation.

The invention also provides for the use of a combination of active ingredient combinations used according to the invention with at least one UVA filter and at least one UVB filter as an antioxidant and for the use of a combination of active ingredients according to the invention with at least one UVA filter and at least one UVB filter as an antioxidant in a cosmetic or dermatological preparation.

Cosmetic and dermatological preparations with an effective content of combinations of active ingredient combinations used according to the invention can also contain inorganic pigments which are normally used in cosmetics for protecting the skin against UV rays. These are oxides of titanium, zinc, zirconium, silicon, manganese, cerium and mixtures thereof, and modifications in which the oxides are the active agents. Particular preference is given to pigments based on titanium dioxide.

These combinations of UVA filter and pigment and preparations which comprise this combination are also provided by the invention. The quantities used may be as stated for the aforementioned combinations.

Cosmetic and dermatological preparations for protecting the hair against UV rays according to the invention are, for example, shampoos, preparations which are applied to the hair when rinsing the hair before or after shampooing, before or after permanent waving, before or after coloring or bleaching, preparations for blow-drying or setting the hair, preparations for coloring or bleaching, a styling and treatment lotion, a hairspray or a permanent wave solution.

The cosmetic and dermatological preparations comprise active ingredients and auxiliaries as are usually used for this type of preparation for hair care and hair treatment. Auxiliaries include preservatives, surface-active substances, antifoams, thickeners, emulsifiers, fats, oils, waxes, organic solvents, bactericides, perfumes, dyes or pigments whose task is to color the hair or the cosmetic or dermatological preparation itself, electrolytes and anti-grease substances.

For the purposes of the present invention, electrolytes are understood as meaning water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably chosen from the group consisting of chlorides, sulfates and hydrogensulfates, phosphates, hydrogenphosphates and linear and cyclic oligophosphates and carbonates and hydrogencarbonates.

Cosmetic preparations in the form of a skin cleanser or shampoo preferably comprise at least one anionic, nonionic or amphoteric surface-active substance, or else mixtures of such substances, the active ingredient combinations used according to the invention in aqueous medium, and auxiliaries usually used for this purpose. The surface-active substances or the mixtures of these substances can be present in the shampoo in a concentration between 1% by weight and 50% by weight.

If the cosmetic or dermatological preparations are in the form of a lotion which is rinsed out and applied, for example, before or after bleaching, before or after shampooing, between two shampooing steps, before or after permanent waving, they are, for example, aqueous or aqueous-alcoholic solutions optionally comprising surface-active substances whose concentrations may be between 0.1 and 10% by weight, preferably between 0.2 and 5% by weight.

A cosmetic preparation in the form of a lotion which is not rinsed out, in particular a lotion for setting the hair, a lotion which is used when blow-drying the hair, a styling and treatment lotion, is generally in the form of an aqueous, alcoholic or aqueous-alcoholic solution, and contains at least one cationic, anionic, nonionic or amphoteric polymer or also mixtures thereof, and also active ingredient combinations used according to the invention in an effective concentration. The amount of polymers used is, for example, between 0.1 and 10% by weight, preferably between 0.1 and 3% by weight.

According to the invention, cosmetic preparations for treating and caring for the hair can be in the form of gels which, in addition to an effective content of active ingredients according to the invention and solvents usually used therefor, preferably water, also contain organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, for example aluminum silicates such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The amount of active ingredient according to the invention in a product intended for hair is preferably from 0.1% by weight to 10% by weight, in particular from 0.5% by weight to 5% by weight, based on the total weight of the product.

Aqueous cosmetic cleansers according to the invention or low-water or water-free cleanser concentrates intended for aqueous cleansing may comprise anionic, nonionic and/or amphoteric surfactants, for example

conventional soaps, e.g. fatty acid salts of sodium

alkyl sulfates, alkyl ether sulfates, alkanesulfonates and alkylbenzenesulfonates

sulfoacetates

sulfobetaines

sarcosinates

amidosulfobetaines

sulfosuccinates

sulfosuccinic monoesters

alkyl ether carboxylates

protein-fatty acid condensates

alkylbetaines and amidobetaines

fatty acid alkanolamides

polyglycol ether derivatives

Cosmetic preparations which are cosmetic skin-cleansing preparations can be in liquid or solid form. In addition to active ingredient combinations used according to the invention, they preferably comprise at least one anionic, nonionic or amphoteric surface-active substance or mixtures thereof, if desired one or more electrolytes and auxiliaries usually used for this purpose. The surface-active substance can be present in the cleansing preparations in a concentration of between 1 and 94% by weight, based on the total weight of the preparations.

Cosmetic preparations in the form of a shampoo preferably comprise, in addition to an effective amount of active ingredient according to the invention, at least one anionic, nonionic or amphoteric surface-active substance or mixtures thereof, if desired an electrolyte according to the invention and auxiliaries which are usually used for this purpose. The surface-active substance can be present in the shampoo in a concentration between 1% by weight and 94% by weight.

The compositions according to the invention comprise, apart from the optional aforementioned surfactants, water and, when required, the additives usual in cosmetics, for example perfume, thickeners, dyes, deodorants, antimicrobial substances, refatting agents, complexing agents and sequestering agents, pearlizing agents, plant extracts, vitamins, active ingredients and the like.

The present invention also covers a cosmetic method of protecting the skin and hair against oxidative or photooxidative processes which comprises applying a cosmetic composition which comprises an effective concentration of active ingredient combinations used according to the invention in a sufficient quantity to the skin or hair.

The amount of active ingredient combinations used according to the invention in these preparations is preferably 0.1-20% by weight, preferably 0.5-10% by weight, in particular 1.0-5.0% by weight, based on the total weight of the preparations.

The invention also provides the process for the preparation of the cosmetic compositions according to the invention, which comprises incorporating active ingredient combinations according to the invention into cosmetic or dermatological formulations in a manner known per se.

The examples below serve to illustrate the present invention without limiting it. Unless stated otherwise, all quantities, proportions and percentages are by weight and based on the total amount or on the total weight of the preparations.

### EXAMPLE 1 (O/W CREAM)

| | % by wt. |
|---|---|
| Glyceryl stearate citrate | 2.00 |
| Stearyl alcohol | 4.00 |
| Caprylic/capric triglycerides | 3.00 |
| Octyldodecanol | 5.00 |
| Hydrogenated coconut fatty acid glycerides | 2.00 |
| Glycerol | 3.00 |
| Carbomer | 0.10 |
| α-Lipoic acid | 0.20 |
| Sodium hydroxide | q.s. |
| Preservative | q.s. |
| Perfume | q.s. |
| Water, demineralized | ad 100.0 |
| pH adjusted to 5.5 | |

### EXAMPLE 2 (O/W LOTION)

| | % by wt. |
|---|---|
| Glyceryl stearate citrate | 3.00 |
| Stearyl alcohol | 2.00 |
| Caprylic/capric triglycerides | 1.00 |
| Dicaprylyl ether | 2.00 |
| Octyldodecanol | 1.00 |
| Glycerol | 3.00 |
| EDTA | 0.20 |
| Carbomer | 0.15 |
| α-Lipoic acid | 0.10 |
| sodium hydroxide | q.s. |
| Preservative | q.s. |
| Perfume | q.s. |
| Water, demineralized | ad 100.0 |
| pH adjusted to 5.0 | |

### EXAMPLE 3 (O/W CREAM)

| | % by wt. |
|---|---|
| Glyceryl stearate citrate | 3.00 |
| Cetyl stearyl alcohol | 4.00 |
| Paraffin oil | 3.00 |
| Caprylic/capric triglycerides | 5.00 |
| Dicaprylyl ether | 5.00 |
| Xanthan gum | 0.10 |
| Citric acid | 0.10 |
| Sodium citrate | 0.20 |
| α-Lipoic acid | 0.50 |
| Glycerol | 3.00 |
| Perfume, preservative, dyes etc. | q.s. |
| Water | ad 100.00 |
| pH adjusted to 6.0 | |

### EXAMPLE 4 (EMULSION MAKE-UP)

| | % by wt. |
|---|---|
| Glyceryl stearate citrate | 3.00 |
| Stearyl alcohol | 1.00 |
| Caprylic/capric triglycerides | 3.00 |
| Dimethicone | 1.00 |
| Glycerol | 3.00 |
| Carbomer | 0.15 |
| Mica | 1.00 |
| Magnesium silicate | 1.00 |
| Iron oxide | 1.00 |
| Titanium dioxide | 2.50 |
| Talc | 5.00 |
| α-Lipoic acid | 0.30 |
| Sodium hydroxide | q.s. |
| Preservative | q.s. |
| Perfume | q.s. |
| Water, demineralized | ad 100.00 |
| pH adjusted to 5.5 | |

### EXAMPLE 5 (O/W CREAM)

| | % by wt. |
|---|---|
| Glyceryl stearate citrate | 3.00 |
| Cetyl stearyl alcohol | 3.00 |
| Octyldodecanol | 3.00 |
| Caprylic/capric triglycerides | 5.00 |
| Squalane | 1.00 |
| Jojoba oil | 1.00 |
| Cyclomethicone | 3.00 |
| Dimethicone | 0.50 |
| Vitamin E acetate | 1.00 |
| Paraffinum liquidum | 1.00 |
| Xanthan gum | 0.10 |
| α-Lipoic acid | 1.00 |
| Retinyl palmitate | 0.20 |
| Glycerol | 3.00 |
| BHT | 0.10 |
| Perfume, preservative, dyes etc. | q.s. |
| Water | ad 100.00 |
| pH adjusted to 6.5 | |

### EXAMPLE 6 (O/W LOTION)

| | % by wt. |
|---|---|
| Glyceryl stearate citrate | 2.00 |
| Stearyl alcohol | 1.00 |
| Octyldodecanol | 1.00 |
| Caprylic/capric triglyceride | 1.00 |
| Tocopheryl acetate | 2.00 |
| Sodium carbomer | 0.10 |
| α-Lipoic acid | 0.30 |
| Glycerol | 3.00 |
| Perfume, preservative, dyes etc, | q.s. |
| Water | ad 100.00 |
| pH adjusted to 5.5 | |

### EXAMPLE 7 (SUNSCREEN CREAM)

| | % by wt. |
|---|---|
| Glyceryl stearate citrate | 3.00 |
| Cetyl stearyl alcohol | 1.00 |
| Octyldodecanol | 3.00 |
| Paraffin oil | 3.00 |
| Dicaprylyl ether | 3.00 |
| Xanthan gum | 0.10 |
| Sodium carbomer | 0.10 |
| Glycerol | 3.00 |
| α-Lipoic acid | 0.15 |
| Ethylhexyl methoxycinnamate | 4.00 |
| Ethylhexyl salicylate | 2.00 |
| Disodium phenyldibenzimidazole tetrasulfonate | 1.00 |
| Titanium dioxide | 1.00 |
| Disodium EDTA | 0.10 |
| Perfume, preservative, dyes, etc. | q.s. |
| Water | ad 100.00 |
| pH adjusted to 6.0 | |

### EXAMPLE 8 (SUNSCREEN LOTION)

| | % by wt. |
|---|---|
| Glyceryl stearate citrate | 3.00 |
| Stearyl alcohol | 2.00 |
| Caprylic/capric triglycerides | 4.00 |
| Dicaprylyl ether | 2.00 |
| Octyldodecanol | 3.00 |
| Glycerol | 3.00 |
| Butylmethoxydibenzoylmethane | 1.00 |
| 4-Methylbenzylidenecamphor | 2.00 |
| Ethylhexyltriazone | 2.00 |
| EDTA | 0.20 |
| Carbomer | 0.15 |
| α-Lipoic acid | 0.10 |
| Sodium hydroxide | q.s. |
| Preservative | q.s. |
| Perfume | q.s. |
| Water, demineralized | ad 100.00 |
| pH adjusted to 5.0 | |

### EXAMPLE 9 (EMULSIFIER GEL)

| | % by wt. |
|---|---|
| Glyceryl stearate citrate | 3.00 |
| Stearyl alcohol | 1.00 |
| Ethanol | 2.00 |
| Aluminum starch octenyl succinate | 0.25 |
| Talc | 0.25 |
| Xanthan gum | 0.10 |
| Retinyl palmitate | 0.20 |
| α-Lipoic acid | 0.05 |
| BHT | 0.04 |
| Na₂H₂EDTA | 0.20 |
| Glycerol | 3.00 |
| Perfume, preservative, dyes, etc. | q.s. |
| Water | ad 100.00 |
| pH adjusted to 5.5 | |

1. A combination of:
(a) one or more partially neutralized esters of monoglycerides and/or diglycerides of saturated fatty acids with citric acid; and
(b) α-lipoic acid.

2. The combination as claimed in claim 1, wherein the partially neutralized ester of monoglycerides and/or diglycerides of saturated fatty acids with citric acid chosen is glyceryl stearate citrate.

3. A cosmetic or dermatological oil-in-water emulsion preparation comprising a combination as claimed in claim 1.

4. The cosmetic or dermatological oil-in-water emulsion preparation as claimed in claim 3, wherein the partially neutralized ester(s) of monoglycerides and/or diglycerides of saturated fatty acids with citric acid is present in the preparation in a concentration of 0.1-20% by weight based on the total weight of the preparation.

5. The cosmetic or dermatological oil-in-water emulsion preparation as claimed in claim 4, wherein the partially neutralized ester(s) of monoglycerides and/or diglycerides of saturated fatty acids with citric acid is present in the preparation in a concentration of 0.5-10% by weight based on the total weight of the preparation.

6. The cosmetic or dermatological oil-in-water emulsion preparation as claimed in claim 5, wherein the partially neutralized ester(s) of monoglycerides and/or diglycerides of saturated fatty acids with citric acid is present in the preparation in a concentration of 1.0-5.0% by weight based on the total weight of the preparation.

7. A method of caring for skin comprising topically applying to said skin an effective amount therefor of a combination according to claim 1.

8. A method of caring for skin comprising topically applying to said skin an effective amount therefor of a cosmetic or dermatological oil-in-water emulsion preparation according to claim 3.
